(19) United States
(12) Patent Application Publication
(10) Pub. No.: US 2005/0214302 A1
(43) Pub. Date: Sep. 29, 2005

(54) Use of emmprin antagonists for the treatment of diseases associated with excessive angiogenesis

(76) Inventors: Marian Nakada, Malvern, PA (US); Li Yan, Berwyn, PA (US); Yi Tang, Drexel Hill, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/808,699

(22) Filed: Mar. 25, 2004

(51) Int. Cl. A61K 39/395

(52) U.S. Cl. 424/155.1

(57) ABSTRACT

A method of using EMMPRIN antagonists to treat pathological processes associated with proliferative diseases, such as cancer, by specifically preventing or inhibiting the ability of new tissue to develop a blood supply. The invention more specifically relates to methods of treating such diseases by the use of EMMPRIN antagonists such as antibodies directed toward EMMPRIN, including specified portions or variants, specific for at least one EMMPRIN protein or fragment thereof, in an amount effective to inhibit angiogenesis.

### BACKGROUND OF THE INVENTION

1. Field of the Invention

The present invention relates to a method of using antagonists of EMMPRIN (Extracellular Matrix Metalloproteinase Inducer) to treat pathological processes associated with proliferative diseases, such as cancer, by specifically preventing or inhibiting the ability of proliferating tissue to develop a blood supply. The invention more specifically relates to methods of treating such diseases by the use of EMMPRIN antagonists such as antibodies directed toward EMMPRIN, including specified portions or variants, specific for at least one protein or fragment thereof, in an amount effective to inhibit angiogenesis.

2. Background of the Invention

EMMPRIN

Angiogenesis is the process of new vessel formation. In adults, angiogenesis occurs only locally and transiently under physiological conditions such as wound healing, menstruation and pregnancy. In contrast, excessive angiogenesis occurs in more than 70 disease conditions such as cancer, atherosclerosis, diabetic blindness, age-related macular degeneration, rheumatoid arthritis, and psoriasis. On the other hand, insufficient angiogenesis underlies diseases such as coronary artery disease, stroke, and delayed wound healing.

Matrix metalloproteinases (MMPs), a family of more than twenty endopeptidases that are capable of cleaving all of the extracellular matrix components, play critical roles in angiogenesis [Klagsbrun and Moses 1999]. Angiogenesis initiates as the breakdown of blood vessel basement membrane by capillary endothelial cells activated by angiogenic stimulators derived from tumors, inflammation sites, or tissues undergo other pathological conditions. The activated endothelial cells express increased MMPs, which in turn, enable disseminated endothelial cells to migrate away from their parental vessels. Only after the cells escape, do they respond to various growth factors to proliferate, and eventually go through a complex differentiation process to form new vessels. Depletion of MMPs, such as MMP-2 or MMP-9, results in a significant inhibition of tumor angiogenesis, supporting the critical role of MMPs in this process. [Bergers et al. 2000; Fang et al. 2000].

Extracellular matrix metalloproteinase inducer (EMMPRIN) (also known as CD 147) is a 58 kDa glycoprotein, originally purified from the plasma membrane of cancer cells and was designated tumor collagenase stimulating factor (TCSF) because of its ability to stimulate collagenase-1 (MMP-1) synthesis by tumor stromal fibroblast cells [Biswas et al. 1995; Ellis et al. 1989]. It was demonstrated to be identical to the M6 antigen and human Basigen (Biswas et al, Cancer Res. 55: 434, 1995). Subsequent studies further demonstrated that EMMPRIN also induced fibroblast synthesis of MMP-2, MMP-3, as well as the membrane-type 1 MMP (MT1-MMP) and MT2-MMP that function as endogenous activator for MMP-2 [Guo et al. 1997; Kataoka et al. 1993; Sameshima et al. 2000b]. Several clinical studies have demonstrated that the expression level of EMMPRIN in tumor tissues is significantly higher than that in peritumoral stromal tissues. These tumors include lung [Polette et al. 1997], breast [Polette et al. 1997], bladder [Javadpour and Guirguis 1992; Muraoka et al. 1993], and glioma [Sameshima et al. 2000a]. Examination of EMMPRIN expression in these clinical samples by a variety of means, including Northern blot, in situ hybridization and immunostaining, revealed that EMMPRIN is expressed by tumor cells, but not by the neighboring stromal cells. On the other hand, MMPs are expressed by peritumoral stromal cells. The role of EMMPRIN in tumor growth and metastasis was directly illustrated using EMMPRIN-overexpressing human breast cancer cells. MDA MB 436 cells are normally slow growing cells when they are implanted into nude mice. However, when these cells were transfected with EMMPRIN, they adopted a more aggressive growth pattern, with both accelerated growth rate and metastatic phenotypes [Zucker et al. 2001].

Disorders Associated with Inappropriate Angiogenesis

Angiogenesis is the process of generating new capillary blood vessels, and it results from activated proliferation of endothelial cells. Neovascularization is tightly regulated, and occurs only during embryonic development, tissue remodeling, wound healing and periodic cycle of corpus luteum development (Folkman and Cotran, Relation of vascular proliferation to tumor growth, Int. Rev. Exp. Pathol. ′16, 207-248(1976)).

Endothelial cells normally proliferate much more slowly than other types of cells in the body. However, if the proliferation rate of these cells becomes unregulated, pathological angiogenesis can result. Pathological angiogenesis is involved in many diseases. For example, cardiovascular diseases such as angioma, angiofibroma, vascular deformity, atherosclerosis, synechia and edemic sclerosis; and opthalmological diseases such as neovascularization after cornea implantation, neovascular glaucoma, diabetic retinopathy, angiogenic corneal disease, macular degeneration, pterygium, retinal degeneration, retrolental fibroplasias, and granular conjunctivitis are related to angiogenesis. Chronic inflammatory diseases such as arthritis; dermatological diseases such as psoriasis, telangiectasis, pyogenic granuloma, seborrheic dermatitis, venous ulcers, acne, rosacea (acne rosacea or erythematosa), warts (verrucas), eczema, hemangiomas, lymphangiogenesis are also angiogenesis-dependent.

Vision can be impaired or lost because of various ocular diseases in which the vitreous humor is infiltrated by capillary blood. Diabetic retinopathy can take one of two forms, non-proliferative or proliferative. Proliferative retinopathy is characterized by abnormal new vessel formation (neovascularization), which grows on the vitreous surface or extends into the vitreous cavity. In advanced disease, neovascular membranes can occur, resulting in a traction retinal detachment. Vitreous hemorrhages may result from neovascularization. Visual symptoms vary. A sudden severe loss of vision can occur when there is intravitreal hemorrhage. Visual prognosis with proliferative retinopathy is more guarded if associated with severe retinal ischemia, extensive neovascularization, or extensive fibrous tissue formation. Macular degeneration, likewise takes two forms, dry and wet. In exudative macular degeneration (wet form), which is much less common, there is formation of a subretinal network of choroidal neovascularization often associated with intraretinal hemorrhage, subretinal fluid, pigment epithelial detachment, and hyperpigmentation. Eventually, this complex contracts and leaves a distinct elevated scar at the posterior pole. Both forms of age-related macular degeneration are often bilateral and are preceded by drusen in the macular region. Another cause of loss of vision related to angiogenic etiologies are damage to the iris. The two most common situations that result in the iris being pulled up into the angle are contraction of a membrane such as in neovascular glaucoma in patients with diabetes or central retinal vein occlusion or inflammatory precipitates associated with uveitis pulling the iris up into the angle (Ch. 99. The Merck Manual 17^{th} Ed. 1999).

Rheumatoid arthritis, an inflammatory disease, also results in inappropriate angiogenesis. The growth of vascular endothelial cells in the synovial cavity is activated by the inflammatory cytokines, and results in cartilage destruction and replacement with pannus in the articulation (Koch A K, Polyerini P J and Leibovich S J, Arth; 15 Rhenium, 29, 471-479(1986); Stupack D G, Storgard C M and Cheresh D A, Braz. J. Med. Biol. Res., 32, 578-581(1999); Koch A K, Arthritis Rheum, 41, 951 962(1998)).

Psoriasis is caused by uncontrolled proliferation of skin cells. Fast growing cell requires sufficient blood supply, and abnormal angiogenesis is induced in psoriasis (Folkman J., J. Invest. Dermatol., 59, 40-48(1972)).

There is now considerable evidence that tumor growth and cancer progression requires angiogenesis, the formation of new blood vessels in order to provide tumor tissue with nutrients and oxygen, to carry away waste products and to act as conduits for the metastasis of tumor cells to distant sites (Folkman, et al. N Engl J Med 285: 1181-1186, 1971 and Folkman, et al. N Engl J Med 333: 1757-1763, 1995).

A number of factors are involved in processes and events leading to angiogenesis: cell adhesion molecules, integrins, vascular endothelial growth factor (VEGF), TNFalpha, bFGF, and cytokines including IL-6 and IL-12. For example, the closely related but distinct integrins αvβ3 and αVβ5 have been shown to mediate independent pathways in the angiogenic process. An antibody generated against αvβ3 blocked basic fibroblast growth factor (bFGF) induced angiogenesis, whereas an antibody specific to αVβ5 inhibited vascular endothelial growth factor (VEGF) induced angiogenesis (Eliceiri, et al., J. Clin. Invest. 103: 1227-1230 (1999); Friedlander et al., Science 270: 1500-1502 (1995)). IL-6 is elevated in tissues undergoing angiogenesis and can induce VEGF in A431 cells, a human epidermoid carcinoma cell line (Cohen, et al. J. Biol. Chem. 271: 736-741, 1996).

Thus, angiogenesis is known to be a contributing factor in a number of pathological conditions including the ability of tumors to grow and metastasize, disorders of the eye including retinopathies, and disorders of the skin including Kaposi's Sarcoma. While numerous factors have been shown to be associated with these processes, it has not heretofore been demonstrated that EMMPRIN directly stimulates VEGF production, stimulates endothelial cells, in addition to local fibroblast cells, to express MMPs and therefore facilitate tumor angiogenesis, growth, invasion and metastasis.

### SUMMARY OF THE INVENTION

The present invention relates to a method of using antagonists of EMMPRIN, including antibodies directed toward EMMPRIN, and specified portions or variants thereof specific for at least one EMMPRIN protein or fragment thereof, to inhibit angiogenesis in disease conditions associated with abnormal angiogenesis. Such EMMPRIN antagonists such as antibodies can act through their ability to prevent the ability of EMMPRIN from stimulating MMP expression by microvascular endothelial cells, the cells involved in angiogenesis, in a dose-dependent fashion. Secondly, such antagonists or antibodies can act by limiting EMMPRIN induction of VEGF in the local environment thereby reducing the angiongenic potential of the tissue. By interfering with angiogenesis, such antagonists can prevent events associated with the initiation or progression of cancer tissue including events involved with angiogenesis and the metastatic spread of cancer. Based on the aforementioned action of the EMMPRIN antagonists of the invention, these antagonists can be best described as anti-angiogenic EMMPRIN antagonists.

Thus, in accordance with the invention, we have, for the first time, demonstrated that EMMPRIN can directly stimulate MMP-1 expression by microvascular endothelial cells, the cells involved in angiogenesis, in a dose-dependent fashion. This stimulation is specifically inhibited by function-blocking anti-EMMPRIN monoclonal antibodies. Since MMPs are essential for angiogenesis, such EMMPRIN antagonists can be useful as therapeutics for such diseases as cancer, diabetic blindness, age-related macular degeneration, rheumatoid arthritis, and psoriasis.

In one embodiment, the EMMPRIN antagonist is an capable of preventing the production of EMMPRIN by cells, such as an siRNA or a shRNA molecule.

In a particular embodiment, the EMMPRIN antagonist is an antibody that specifically binds EMMPRIN. A particular advantage of such antibodies is that they are capable of binding EMMPRIN in a manner that prevents its action systemically. The method of the present invention thus employs antibodies having the desirable neutralizing property which makes them ideally suited for therapeutic and preventative treatment of metastatic disease states associated with various forms of cancer in human or nonhuman patients. Accordingly, the present invention is directed to a method of treating a disease or condition which is dependent on angiogenesis in a patient in need of such treatment which comprises administering to the patient an amount of a neutralizing EMMPRIN antibody to inhibit angiogenesis.

In a particularly preferred embodiment of the EMMPRIN antagonist antibody of the invention, the antibody is known as CNTO146 and is a murine anti-human EMMPRIN of the IgG1k class which has distinguished capability of inhibiting EMMPRIN-induced MMP production, including inhibiting MMP-1 production in fibroblast stimulated with recombinant EMMPRIN, as well as inhibiting MMP production in the co-culture of tumor cells and fibroblast cells.

### BRIEF DESCRIPTION OF THE DRAWINGS

FIG. 1. Schematic illustration of the central role of EMMPRIN in diseases involving abnormal angiogenesis.

FIG. 2. Recombinant EMMPRIN dose-dependently stimulated MMP-1 production by HMVEC-L cells.

FIG. 3. Inhibition of EMMPRIN-induced MMP-1 production in HMVEC-L cells by a neutralizing anti-EMMPRIN monoclonal antibody.

FIG. 4. is a set of bar graphs showing (A) the relative endothelial cell migration induced by FIG. 5 is a bar graph showing the average final tumor weights of tumors produced by MDA MB231 human breast tumor cells manipulated to express greater or lesser amounts of EMMPRIN than normal (WT) and (B) is a bar graph showing the relative reduction in the migration of endothelial cells induced by WT cells in the presence of increasing concentrations of anti-VEGF antibody.

FIG. 5A is a bar graph showing the average final tumor weights of tumors produced by MDA MB231 human breast tumor cells manipulated to express greater (S1-3) or lesser (AS1-5) and (AS2-5) amounts of EMMPRIN than normal (WT) or Vector control cells. 5B is a micrograph showing the difference in angiogenic structures between tumors produced by implantation of mice with WT versus S1-3 cells. 5C is a set of bar graphs shoinw the amount of human VEGF (left panel) and mouse VEGF (right panel) in tumors produced by MDA MB231 human breast tumor cell types.

FIG. 6A is a bar graph showing the amount of human EMMMPRIN in tissue extracts from xenograft tumors derived from WT, Vector control, S1-3, or AS EMMPRIN engineered human tumor cells. B. is a photo of a zymography gel showing MMP expression profile in tissue extracts from the same tumors containing where 10 μg of total protein was added to each lane. C. is a bar graph showin the quantitative determination of human and mouse MMP-2 levels in xenograft tumors. D. is a pair of bar graphs showing quantitative determination of human (left panel) and mouse (right panel) MMP-9 levels in xenograft tumors.

FIG. 7. Photographs showing increased angiogenesis evidenced by numerous new capillary blood vessels in tumors derived from sense cells expressing EMMPRIN, but not in tumors derived from WT OR AS cells.

FIG. 8 shows photographs of tumors after immunohistochemical analysis of MMP, VEGF, EMMPRIN: A. H&E staining of MDA-MB-231 xenograft tumors; B. Mouse MMP-9 staining; C. Mouse EMMPRIN staining; D. Blood vessel staining with anti-CD31 antibodies. Left panels—Vector control tumors; right panels—S1-3 tumors.

### DETAILED DESCRIPTION OF THE INVENTION

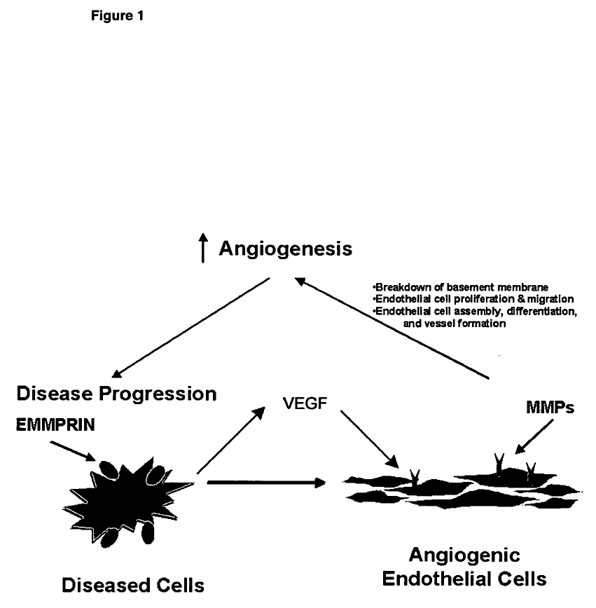

EMMPRIN expressed by cells in diseased tissues directly stimulates neighboring endothelial cells, which results in an increase in MMP expression, i.e., MMP-1. (See FIG. 1) These MMPs, in turn, mediate the breakdown of basement membrane of existing blood vessels; promote endothelial cells to migrate away from parental vessels; stimulate the expression and release of angiogenic growth factors; enable endothelial cells to respond to angiogenesis stimulatory factors leading to cell proliferation; and facilitate the remodeling of extracellular matrix for endothelial cell differentiation and assembly of new vessels. All these changes lead to an increase in angiogenesis and further contribute to the overall disease progression.

The anti-angiogenic EMMPRIN antagonists of the invention are useful in inhibiting and preventing angiogenesis in so far as they block the stimulatory effects of EMMPRIN on endothelial cells, reduce VEGF production by endothelial cell, reduce endothelial cell division, decrease endothelial cell migration, and impair the activity of the proteolytic enzymes secreted by the endothelium. A number of pathologies including various forms of solid primary tumors and the metastases, lesions of the eye and disorders of the skin are improved by treatment with EMMPRIN antagonists in the method of the present invention.

Cancer

Both benign and malignant tumors, including various cancers such as, cervical, anal and oral cancers, stomach, colon, bladder, rectal, liver, pancreatic, lung, breast, cervix uteri, corpus uteri, ovary, prostate, testis, renal, brain/cns (e.g., gliomas), head and neck, eye or ocular, throat, skin melanoma, acute lymphocytic leukemia, acute myelogenous leukemia, Ewing's Sarcoma, Kaposi's Sarcoma, basal cell carinoma and squamous cell carcinoma, small cell lung cancer, choriocarcinoma, rhabdomyosarcoma, angiosarcoma, hemangioendothelioma, Wilms Tumor, neuroblastoma, mouth/pharynx, esophageal, larynx, kidney and lymphoma, among others may be treated using anti-EMMPRIN antibodies of the present invention. In addition, conditions such as neurofibromatosis, tuberous sclerosis (each of which conditions produces benign tumors of the skin), hemangiomas and lymphangiogenesis, among others, may be treated effectively with EMMPRIN antagonists according to the present invention.

A secondary tumor, a metastasis, is a tumor which originated in a primary site elsewhere in the body, but has now spread to a distant organ. The common routes for metastasis are direct growth into adjacent structures, spread through the vascular or lymphatic systems, and tracking along tissue planes and body cavaties with, for example, peritoneal fluid or cerebrospinal fluid. Secondary hepatic tumors are one of the most common causes of death in cancer patients and are by far and away the most common form of liver tumor. Although virtually any malignancy can metastasize to the liver, tumors which are most likely to spread to the liver include: cancer of the stomach, colon, and pancreas; melanoma; tumors of the lung, oropharynx, and bladder; Hodgkin's and non-Hodgkin's lymphoma; tumors of the breast, ovary, and prostate. Secondary lung, brain, and bone tumors are common to advanced stage breast, prostate and lung cancers. Any cancer may metastasize to bone, but metastases from carcinomas are the most common, particularly those arising in the breast, lung, prostate, kidney, and thyroid. Carcinoma of the lung is very commonly accompanied by hematogenous metastatic spread to the liver, brain, adrenals, and bone and may occur early, resulting in symptoms at those sites before obvious pulmonary symptom. Metastases to the lungs are common from primary cancers of the breast, colon, prostate, kidney, thyroid, stomach, cervix, rectum, testis, and bone and from melanoma. Each one of the above-named secondary tumors may be treated by the antibodies of the present invention.

In addition to tumors, numerous other non-tumorigenic angiogeneis-dependent diseases which are characterized by the abnormal growth of blood vessels may also be treated with the anti-angiogenic EMMPRIN antagonists of the present invention.

Representative examples of such non-tumorigenic angiogenesis-dependent diseases include corneal neovascularization, hypertrophic scars and keloids, proliferative diabetic retinopathy, rheumatoid arthritis, arteriovenous malformations (discussed above), atherosclerotic plaques, delayed wound healing, hemophilic joints, nonunion fractures, Osler-Weber syndrome, psoriasis, pyogenic granuloma, scleroderma, tracoma, menorrhagia (discussed above) and vascular adhesions.

Angiogenic Conditions of the Eyes

The cornea is a tissue which normally lacks blood vessels. In certain pathological conditions, however, capillaries may enter the cornea from the pericorneal vascular plexus of the limbus. When the cornea becomes vascularized, it also becomes clouded, resulting in a decline in the patient's visual acuity. Visual loss may become complete if the cornea completely opacitates.

Blood vessels can enter the cornea in a variety of patterns and depths, depending upon the process which incites the neovascularization. These patterns have been traditionally defined by ophthalmologists in the following types: pannus trachomatosus, pannus leprosus, pannus phylctenulosus, pannus degenerativus, and glucomatous pannus. The corneal stroma may also be invaded by branches of the anterior ciliary artery (called interstitial vascularization) which causes several distinct clinical lesions: terminal loops, a “brush-like” pattern, an umbel form, a lattice form, interstitial arcades (from episcleral vessels), and aberrant irregular vessels.

Corneal neovascularization can result from corneal ulcers. A wide variety of etiologies may produce corneal ulcers including for example corneal infections (trachoma, herpes simplex keratitis, leishmaniasis and onchocerciasis), immunological processes (graft rejection and Stevens-Johnson's syndrome), alkali burns, trauma, inflammation (of any cause), toxic and Vitamin A or protein deficiency states, and as a complication of wearing contact lenses.

While the cause of corneal neovascularization may vary, the response of the cornea to the insult and the subsequent vascular ingrowth is similar regardless of the cause. Several angiogenic factors are likely involved in this process, many of which are products of the inflammatory response. Indeed neovascularization of the cornea appears to only occur in association with an inflammatory cell infiltrate, and the degree of angiogenesis is proportional to the extent of the inflammatory reaction. Corneal edema further facilitates blood vessel ingrowth by loosening the corneal stromal framework through which the capillaries grow.

Topical therapy with EMMPRIN antibodies may also be useful prophylactically in corneal lesions which are known to have a high probability of inducing an angiogenic response (such as chemical burns). In these instances the treatment, likely in combination with steroids, may be instituted immediately to help prevent subsequent complications.

Such methods may also be utilized in a similar fashion to prevent capillary invasion of transplanted corneas. Use in combination with a steroid is also contemplated.

Neovascular glaucoma is a pathological condition wherein new capillaries develop in the iris of the eye. The angiogenesis usually originates from vessels located at the pupillary margin, and progresses across the root of the iris and into the trabecular meshwork. Fibroblasts and other connective tissue elements associate with the capillary growth and a fibrovascular membrane develops which spreads across the anterior surface of the iris eventually forming a scar. The scar formation prevents adequate drainage of aqueous humor resulting in an increase in intraocular pressure that may result in blindness.

Neovascular glaucoma generally occurs as a complication of diseases in which retinal ischemia is predominant. In particular, about one third of the patients with this disorder have diabetic retinopathy. Other causes include chronic retinal detachment, end-stage glaucoma, carotid artery obstructive disease, retrolental fibroplasia, sickle-cell anemia, intraocular tumors, and carotid cavernous fistulas.

Angiogenic Conditions of the Skin

Within another aspect of the present invention, methods are provided for treating hypertrophic scars and keloids, comprising the step of administering one of the above-described anti-angiogenic compositions to a hypertrophic scar or keloid.

Healing of wounds and scar formation occurs in three phases: inflammation, proliferation, and maturation. The first phase, inflammation, occurs in response to an injury which is severe enough to cause tissue damage and vascular leaking. During this phase, which lasts 3 to 4 days, blood and tissue fluid form an adhesive coagulum and fibrinous network which serves to bind the wound surfaces together. This is then followed by a proliferative phase in which there is ingrowth of capillaries and connective tissue from the wound edges, and closure of the skin defect. Finally, once capillary and fibroblastic proliferation has ceased, the maturation process begins wherein the scar contracts and becomes less cellular, less vascular, and appears flat and white. This final phase may take between 6 and 12 months.

Overproduction of connective tissue at the wound site causes a persistently cellular and possible red and raised scar to be formed. If the scar remains within the boundaries of the original wound it is referred to as a hypertrophic scar, but if it extends beyond the original scar and into the surrounding tissue, the lesion is referred to as a keloid. Hypertrophic scars and keloids are produced during the second and third phases of scar formation. Several wounds are particularly prone to excessive endothelial and fibroblastic proliferation, including burns, open wounds, and infected wounds. With hypertrophic scars, some degree of maturation occurs and gradual improvement occurs. In the case of keloids however, an actual tumor is produced which can become quite large. Spontaneous improvement in such cases rarely occurs. Administration of an anti-EMMPRIN antibody in the method of the present invention to inhibit angiogenesis in such conditions can thus inhibit the formulation of such keloid scars.

Anti-Angiogenic Combinations with EMMPRIN Antagonists

Angiogenesis is characterized by the invasion, migration and proliferation of smooth muscle and endothelial cells. The αvβ3 integrin (also known as the vitronectin receptor) is known to play a role in various conditions or disease states including tumor metastasis, solid tumor growth (neoplasia), osteoporosis, Paget's disease, humoral hypercalcemia of malignancy, angiogenesis, including tumor angiogenesis, retinopathy, including macular degeneration, arthritis, including rheumatoid arthritis, periodontal disease, psoriasis and smooth muscle cell migration (e.g. restenosis).

The adhesion receptor integrin αvβ3 binds vitronectin, fibrinogen, von Willebrand Factor, laminin, thrombospondin, and other like ligands. It was identified as a marker of angiogenic blood vessels in chick and man and plays a critical role in angiogenesis or neovascularization. Antagonists of αvβ3 inhibit this process by selectively promoting apoptosis of cells in neovasculature. Therefore, αvβ3 antagonists would be useful therapeutic targets for treating such conditions associated with neovascularization (Brooks et al., Science, Vol. 264, (1994), 569-571). Additionally, tumor cell invasion occurs by a three step process: 1) tumor cell attachment to extracellular matrix; 2) proteolytic dissolution of the matrix; and 3) movement of the cells through the dissolved barrier. This process can occur repeatedly and can result in metastases at sites distant from the original tumor. The αvβ3 integrin has been shown to play a role in tumor cell invasion as well as angiogenesis.

As the antagonists of αvβ3 and neutralizing anti-EMMPRIN antibodies both target neovasculature but act through different mechanisms, the combination of anti-integrin antibodies with anti-EMMPRIN antibodies should result in a particularly potent and effective combination therapy with little normal tissue toxicity. Thus, in one embodiment of the present invention, there is provided a method of treating a disease or condition associated with angiogenesis which comprises administering a combination of an integrin antagonist and an anti-EMMPRIN antibody to inhibit angiogenesis in a patient in need of such treatment. Other antibodies which selectively bind integrins or integrin subunits, especially those that bind the alphaV subunit, are disclosed in U.S. Pat. Nos. 5,985,278 and 6,160,099. Mabs that inhibit binding of alphaVbeta3 to its natural ligands containing the tripeptide argininyl-glycyl-aspartate (RGD) are disclosed in U.S. Pat. No. 5,766,591 and WO0078815.

A preferred combination of antibodies is the anti-alphaVbeta3 and anti-alphaVbeta5 Mab described in applicant's co-pending application U.S. Ser. No. 09/092,026 and an anti-EMMPRIN antibody, as disclosed herein. Both of the foregoing applications are incorporated by reference into the present application and form part of the disclosure hereof. In accordance with the invention, other known anti-angiogenesis agents such as thalidomide may also be employed in combination with an anti-EMMPRIN antibody.

Methods of Evaluating Anti-Angiogenic Activity

Widely accepted functional assays of angiogenesis and, hence, anti-angiogenic agents are the chick chorio-allantoic membrane assay (CAM) assay and the corneal micropocket assay of neovascularization.

For the CAM assay, fertilized chick embryos are removed from their shell on day 3 (or 4) and incubated in a Petri dish in high humidity and 5% CO₂. On day 6, a methylcellulose disc (10 microL) containing the test substance is implanted on the chorioallantoic membrane. The embryos were examined 48 hours later, and if a clear avascular zone appears around the methylcellulose disc, the diameter of that zone is measured. The larger the zone, the more effective the antibody. India ink can be injected into the heart of some embryos just before formalin fixation so that vessels are visible near the edge of the avascular zone in histological sections. Histologic cross-sections of the chorioallantoic are examined to determine whether the test substance prevents normal development of the capillaries. This method, described in U.S. Pat. No. 5,001,116 which is also specifically incorporated herein by reference, showed the test useful in the selection of anti-angiogenic compounds or combinations of compounds.

The corneal micropocket assay of neovascularization may be practiced using rat or rabbit corneas. This in vivo model is widely accepted as being generally predictive of clinical effect, as described in many review articles and papers such as O'Reilly et. al. Cell 79: 315-328.

Briefly, a plug or pellet containing the recombinant bFGF (Takeda Pharmaceuticals-Japan) is implanted into corneal micropockets of each eye of an anesthetized female New Zealand white rabbit, 2 mm from the limbus followed by topical application of erythromycin ointment onto the surface of the cornea. The animals are dosed with the test compounds and examined with a slit lamp every other day by a corneal specialist. Various mathematical models are utilized to determine the amount of vascularized cornea and this formula was found to provide the most accurate approximation of the area of the band of neovascularization that grows towards the pellet.

The method may also be practiced using rats.

In the present invention, the corneal micropocket assay may be used to demonstrate the anti-angiogenesis effect of anti-EMMPRIN antibodies. This is evidenced by a significant reduction in angiogenesis, as represented by a consistently observed and preferably marked reduction in the number of blood vessels within the cornea.

Endothelial and Non-Endothelial Cell Proliferation

It is important to establish which cell types are involved in the angiogenic processes specific for tumor vascularization. Tumor vessels are generally primitive, that is, contain only endothelial cells. Other cell types found in more mature vessels include: smooth muscle cells, retinal pigment epithelial cells, fibroblasts, and epithelial cells, as well as tumor cells such as hemangioendothelioma cells or carcinoma cells. One example of an angiogenesis inhibitor that specifically inhibits endothelial cell proliferation is ANGIOSTATIN® protein. (O'Reilly et al., 1994 supra).

Various representative cell lines are available for testing. Bovine aortic smooth muscle (SMC), bovine retinal pigment epithelial (RPE), mink lung epithelial (MLE), Lewis lung carcinoma (LLC), and EOMA hemangioendothelioma cells and 3T3 fibroblasts. For the proliferation assays, cells are washed with PBS and dispersed in a 0.05% solution of trypsin. Optimal conditions for the cell proliferation assays are established for each different cell type. Generally, cells are trypsinized and re-seeded in growth medium in the presence and absence of EMMPRIN and anti-EMMPRIN neutralizing Mab. After approximately 72 hours, the change in cell number is assessed by using a vital stain such as a tetrazolium dye or by LDH release (Promega, Madison Wis.) or by individual cell counting.

EMMPRIN Antagonists

As used herein, the term “EMMPRIN antagonists” refers to a substance which inhibits or neutralizes the angiogenic activity of EMMPRIN. Such antagonists accomplish this effect in a variety of ways. One class of EMMPRIN antagonists will bind to EMMPRIN protein with sufficient affinity and specificity to neutralize the angiogenic effect of EMMPRIN. Included in this class of molecules are antibodies and antibody fragments (such as for example, F(ab) or F(ab′)₂ molecules). Another class of EMMPRIN antagonists are fragments of EMMPRIN protein, muteins or small organic molecules i.e. peptidomimetics, that will bind to EMMPRIN or EMMPRIN binding partners, thereby inhibiting the angiogenic activity of EMMPRIN. The EMMPRIN antagonist may be of any of these classes as long as it is a substance that inhibits EMMPRIN angiogenic activity. EMMPRIN antagonists include EMMPRIN antibody, EMMPRIN receptor antibody, modified EMMPRIN, antisense EMMPRIN and partial peptides of EMMPRIN or EMMPRINR.

Anti-EMMPRIN Antibodies

Neutralizing antibodies to soluble factors that mediate inflammation and tumor proliferation, such as TNFalpha, have proved to highly effective therapeutics. REMICADE (infliximab) sold by Centocor, Malvern, Pa. an anti-TNFalpha MAb is prescribed for RA and Crohn's Disease and RITUXAN (rituximab) an anti-CD20 Mab sold by Genentech, San Bruno, Calif. is used to treat B-cell lymphoma. “Neutralizing” Mabs not only bind their target but also inhibit its biological activity, usually by preventing its interaction with its cognate cell surface receptor. In certain cases, the target protein will comprise more than one active domain and exhibit multiple actions due to binding to more than one ligand or receptor. EMMPRIN is such a molecule and exhibits two immunoglobulin-like domains in the extracellular portion of the molecule, the Ig-like C2-type domain from aa 22-103 of basigin isoform 2 (NCBI accession # NP_{—}940991) domain and the Ig-like V-type domain at 105-199 of the same isoform (Biswas, Zhang, DeCastro, Guo, Nakamura, Kataoka and Nabeshima, (1995), Cancer Res 55: 434-9). Monoclonal antibodies raised to EMMPRIN from cancer cells are capable of inhibiting EMMPRIN-induced MMP production in fibroblast cells, indicating neutralizing activity (Ellis, Nabeshima and Biswas, (1989), Cancer Res 49: 3385-91). These antibodies were subsequent shown to bind to EMMPRIN in the region 34-99 which lies within the C2-type domain. In contrast, CBL1, a murine IgM, anti-human lymphoblastoid monoclonal antibody that was raised in Balb/c mice immunized with the T cell acute lymphoblastic leukemia cell line (T-ALL) CEM. The latter MAb has been tested clinically in patients with graft versus host disease (Heslop, H. et al. (1995) Lancet 346: 805-806). WO9945031 teaches that antibodies with activities similar to CBL1 share a consensus binding sequence located in a region more C-terminal than the V-type domain, that is RVSR (residues 201-204 of NP_{—}940991) and of a panel of MAbs made to the extracellular domain of EMMPRIN only one, designated M-6/6, is capable of inhibiting OKT3-induced T-cell activation and binds to a region in the C2-type domain Koch, C. et al. (1999) Internat. Immunol. 11: 777-786; Staffler, G. et al. (2003) J. Immunol. 171: 1707-1714). Therefore, selection of a uniquely anti-angiogenic anti-EMMPRIN Mab can be achieved by using a specific set of in vitro assays as screening tools.

Any of the anti-EMMPRIN antibodies known in the art which are anti-angiongenic EMMPRIN antagonists may be employed in the method of the present invention. Murine monocolonal antibodies to EMMPRIN are known as in, for example, in Ellis et al, 1989 supra and Koch, et al. 1999 Internat. Immunol. 11 (5): 777-786.

Accordingly, as used herein, an “EMMPRIN antibody”, “anti-EMMPRIN antibody,” “anti-EMMPRIN antibody portion,” or “anti-EMMPRIN antibody fragment” and/or “anti-EMMPRIN antibody variant” and the like include any protein or polypeptide containing molecule that comprises at least a portion of an immunoglobulin molecule, such as but not limited to at least one complementarity determining region (CDR) of a heavy or light chain or a ligand binding portion thereof, a heavy chain or light chain variable region, a heavy chain or light chain constant region, a framework region, or any portion thereof, or at least one portion of an EMMPRIN binding protein derived from a EMMPRIN protein or peptide, which can be incorporated into an antibody for use in the present invention. Such antibody optionally further affects a specific ligand, such as but not limited to where such antibody modulates, decreases, increases, antagonizes, agonizes, mitigates, alleviates, blocks, inhibits, abrogates and/or interferes with EMMPRIN angiogenic activity, in vitro, in situ and/or in vivo. As a non-limiting example, a suitable anti-EMMPRIN antibody, specified portion or variant of the present invention can bind at least one EMMPRIN protein or peptide, or specified portions, variants or domains thereof. A suitable anti-EMMPRIN antibody, specified portion, or variant affects EMMPRIN angiogenic function in a variety of ways, such as but not limited to, RNA, DNA or protein synthesis, EMMPRIN release, EMMPRIN receptor signaling, EMMPRIN receptor binding, EMMPRIN production and/or synthesis. The term “antibody” is further intended to encompass antibodies, digestion fragments, specified portions and variants thereof, including antibody mimetics or comprising portions of antibodies that mimic the structure and/or function of an antitbody or specified fragment or portion thereof, including single chain antibodies and fragments thereof. Functional fragments include antigen-binding fragments that bind to a mammalian EMMPRIN. For example, antibody fragments capable of binding to EMMPRIN or portions thereof, including, but not limited to Fab (e.g., by papain digestion), Fab′ (e.g., by pepsin digestion and partial reduction) and F(ab′)2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, Immunology, supra).

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)2 heavy chain portion can be designed to include DNA sequences encoding the CH1 domain and/or hinge region of the heavy chain. The various portions of antibodies can be joined together chemically by conventional techniques, or can be prepared as a contiguous protein using genetic engineering techniques.

The anti-EMMPRIN antibody may be a primate, rodent, or human antibody or a chimeric or humanized antibody. As used herein, the term “human antibody” refers to an antibody in which substantially every part of the protein (e.g., CDR, framework, CL, CH domains (e.g., CH1, CH2, CH3), hinge, (VL, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies of the invention can include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, a Fv can comprise a linker peptide, such as 2 to about 8 glycine or other amino acid residues, which connects the variable region of the heavy chain and the variable region of the light chain. Such linker peptides are considered to be of human origin.

Bispecific, heterospecific, heteroconjugate or similar antibodies can also be used that are monoclonal, preferably human or humanized, antibodies that have binding specificities for at least two different antigens. In the present case, one of the binding specificities is for at least one EMMPRIN protein, the other one is for any other antigen. Methods for making bispecific antibodies are known in the art. Traditionally, the recombinant production of bispecific antibodies is based on the co-expression of two immunoglobulin heavy chain-light chain pairs, where the two heavy chains have different specificities (Milstein and Cuello, Nature 305:537 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed, e.g., in WO 93/08829, U.S. Pat. Nos. 6,210,668, 6,193,967, 6,132,992, 6,106,833, 6,060,285, 6,037,453, 6,010,902, 5,989,530, 5,959,084, 5,959,083, 5,932,448, 5,833,985, 5,821,333, 5,807,706, 5,643,759, 5,601,819, 5,582,996, 5,496,549, 4,676,980, WO 91/00360, WO 92/00373, EP 03089, Traunecker et al., EMBO J. 10:3655 (1991), Suresh et al., Methods in Enzymology 121:210 (1986), each entirely incorporated herein by reference.

Anti-EMMPRIN antibodies useful in the methods and compositions of the present invention can optionally be characterized by high affinity binding to EMMPRIN and optionally and preferably having low toxicity. In particular, an antibody, specified fragment or variant of the invention, where the individual components, such as the variable region, constant region and framework, individually and/or collectively, optionally and preferably possess low immunogenicity, is useful in the present invention. The antibodies that can be used in the invention are optionally characterized by their ability to treat patients for extended periods with measurable alleviation of symptoms and low and/or acceptable toxicity. Low or acceptable immunogenicity and/or high affinity, as well as other suitable properties, can contribute to the therapeutic results achieved. “Low immunogenicity” is defined herein as raising significant HAHA, HACA or HAMA responses in less than about 75%, or preferably less than about 50% of the patients treated and/or raising low titres in the patient treated (less than about 300, preferably less than about 100 measured with a double antigen enzyme immunoassay) (Elliott et al., Lancet 344:1125-1127 (1994), entirely incorporated herein by reference).

Suitable antibodies include those that compete for binding to human EMMPRIN with the commercially available monoclonal antibody CD147-RDI/clone UM-8D6 (Research Diagnostics, Inc., Flanders, N.J.).

Compositions and Their Uses

In accordance with the invention, the neutralizing anti-EMMPRIN monoclonal antibodies described herein can be used to inhibit angiogenesis and thus prevent or impair tumor growth and prevent or inhibit metastases. Additionally, such monoclonal antibodies can be used to inhibit angiogenic inflammatory diseases amenable to such treatment, which may include but are not limited to rheumatoid arthritis, diabetic retinopathy, psoriasis, and macular degeneration. The individual to be treated may be any mammal and is preferably a primate, a companion animal which is a mammal and most preferably a human patient. The amount of monoclonal antibody administered will vary according to the purpose it is being used for and the method of administration.

The anti-angiogenic anti-EMMPRIN antibodies may be administered by any number of methods that result in an effect in tissue in which angiogenesis is desired to be prevented or halted. Further, the anti-antiangiongenic anti-EMMPRIN antibodies need not be present locally to impart an anti-angiogenic effect, therefore, they may be administered wherever access to body compartments or fluids containing EMMPRIN is achieved. In the case of inflamed, malignant, or otherwise compromised tissues, these methods may include direct application of a formulation containing the antibodies. Such methods include intravenous administration of a liquid composition, transdermal administration of a liquid or solid formulation, oral, topical administration, or interstitial or inter-operative administration. Adminstration may be affect by the implantation of a device whose primary function may not be as a drug delivery vehicle as, for example, a vascular stent.

In particular, within one aspect of the present invention methods are provided for treating corneal neovascularization (including corneal graft neovascularization), comprising the step of administering a therapeutically effective amount of an anti-angiogenic EMMPRIN antibody of the invention directly to the cornea or systemically to the patient, such that the formation of blood vessels is inhibited.

Within another aspect of the present invention methods are provided for treating neovascular glaucoma, comprising the step of administering a therapeutically effective amount of an anti-angiogenic neutralizing anti-EMMPRIN antibodies directly to the eye or systemically to the patient, such that the formation of blood vessels is inhibited.

In another embodiment of the present invention either an anti-angiogenic EMMPRIN antibody of the invention alone, or in combination with another anti-angiogenic agent are directly injected into a hypertrophic scar or keloid in order to prevent the progression of these lesions. This therapy is of particular value in the prophylactic treatment of conditions which are known to result in the development of hypertrophic scars and keloids such as burns. Therapy may be effective when begun after the proliferative phase has had time to progress (approximately 14 days after the initial injury), but before hypertrophic scar or keloid development.

Administration may also be oral or by local injection into a tumor or tissue but generally, the monoclonal antibody is administered intravenously. Generally, the dosage range is from about 0.05 mg/kg to about 12.0 mg/kg. This may be as a bolus or as a slow or continuous infusion which may be controlled by a microprocessor controlled and programmable pump device.

Alternatively, DNA encoding preferably a fragment of said monoclonal antibody may be isolated from hybridoma cells and administered to a mammal. The DNA may be administered in naked form or inserted into a recombinant vector, e.g., vaccinia virus in a manner which results in expression of the DNA in the cells of the patient and delivery of the antibody.

The monoclonal antibody used in the method of the present invention may be formulated by any of the established methods of formulating pharmaceutical compositions, e.g. as described in Remington's Pharmaceutical Sciences, 1985. For ease of administration, the monoclonal antibody will typically be combined with a pharmaceutically acceptable carrier. Such carriers include water, physiological saline, or oils.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Except insofar as any conventional medium is incompatible with the active ingredient and its intended use, its use in any compositions is contemplated.

The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use.

Abbreviations
Abs antibodies, polyclonal or monoclonal
aV integrin subunit alpha V
b3 integrin subunit beta 3
bFGF basic fibroblast growth factor
IFN interferon
Ig immunoglobulin
IgG immunoglobulin G
IL interleukin
MMP-1
EMMPRIN extracellular matric metalloproteinase inducer
EMMPRINR receptor
sEMMPRINR soluble EMMPRIN receptor
Mab monoclonal antibody
VEGF vascular endothelial growth factor
MMP matrix metallopoteinase

While having described the invention in general terms, the embodiments of the invention will be further disclosed in the following examples.

### EXAMPLE 1

### Recombinant EMMPRIN Stimulates MMP-1 Production by Human Microvascular Endothelial Cells from the Lung (HMVEC-L)

The effect of EMMPRIN on endothelial cells was investigated using microvascular endothelial cells, cells that are directly involved in angiogenesis process in vivo.

HMVEC-L cells were obtained from Clonetics, Walkersville, Md. (Cat# CC-2527, Lot# 8F1528). HMVEC-L cells were cultured under conditions recommended by the supplier. Briefly, cells were cultured in Endothelium Cell Growth Medium MV (EGM-2 MV, Clonetics, Cat#CC-3202) containing human epithelial growth factor (hEGF), hydrocortisone, human basic fibroblast growth factor (hFGF-B), vascular endothelial growth factor (VEGF), human insulin-like growth factor-1 (hIGF-1), ascorbic acid, gentamicin, 5% FBS, at 37° C., 5% CO₂.

Early passage cells (less than passage 3) were trypsinized and washed with RPMI-1640 once. Cells were resuspended in Dilution Medium (DM−Fibroblast Basic Medium+2% FBS) at a concentration of 5×100,000 cells/ml. 100 μl of the cell suspension containing 50,000 cells was added into each well in a 96 well cell culture plate. These wells were preloaded with soluble recombinant human EMMPRIN with final concentrations of 20 μg/ml, 6.67 μg/ml, 2.22 μg/ml, 0.74 μg/ml, 0.25 μg/ml, 0.08 μg/ml, and 0 μg/ml. Cells were incubated at 37° C., 5% CO₂, in a humidified incubator for 1 day and 3 days. Conditioned medium was collected from each well and subjected to MMP-1 activity assay.

Quantitative detection of MMP-1 activity in the conditioned medium was carried out using Human MMP-1 Activity Kit (R&D Systems, Minneapolis, Minn.) (Cat#F1M00). Briefly, MMP-1 in 150 μl of standard or sample was captured by anti-MMP-1 antibodies immobilized at the bottom of each well. Captured MMP-1 was subsequently activated by 4-aminophenylmercuric acetate (APMA). MMP substrate added into each well was cleaved by active MMP-1 and the resulting fluorescence was determined using SpectraFluor Plus Plate Reader (TECAN, Zurich, Switzerland) (Cat# F129005, Ser# 94747) with the following parameters: excitation wavelength at 320 nm and emission wavelength at 405 nm.

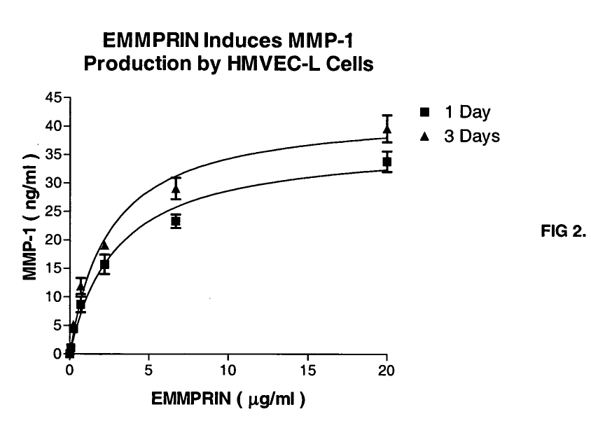

HMVEC-L cells were challenged with different concentrations of recombinant EMMPRIN to stimulate MMP-1 production. As shown in FIG. 2, EMMPRIN dose-dependently stimulated MMP-1 production in endothelial cells. HMVEC-L cells produced approximately 40 ng/ml MMP-1 when treated with 20 μg/ml EMMPRIN. This response of HMVEC-L to EMMPRIN stimulation was even stronger than that by NHLF cells, which produced only half of that amount of MMP-1 in response to the same treatment. The stimulation of MMP-1 production was first observed after one-day challenge and sustained for at least three days.

Results shown in FIG. 2, for the first time, demonstrated that EMMPRIN can directly stimulate MMP-1 expression by microvascular endothelial cells, the cells directly involved in angiogenesis, in a dose-dependent fashion.

### EXAMPLE 2

### Inhibition of EMMPRIN-Induced MMP-1 Production by an Anti-EMMPRIN mAb in HMVEC-L Cells

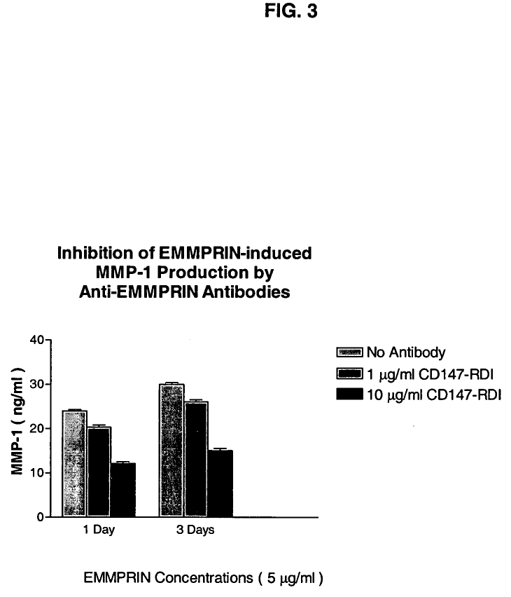

To further confirm the specificity of EMMPRIN-induced MMP-1 production, monoclonal antibodies against human EMMPRIN were included in the assay 15 minutes after cells were stimulated with EMMPRIN. At 10 μg/ml, the CD147-RDI/clone UM-8D6 (Research Diagnostics, Inc., Flanders, N.J.) significantly inhibited MMP-1 production by fibroblast cells induced by EMMPRIN (5 μg/ml) (FIG. 3). However, the other anti-EMMPRIN mAb (mouse anti-human CD147/EMMPRIN, clone HIM6, BD Pharmingen, San Diego, Calif.) was not able to inhibit MMP-1 production induced by EMMPRIN.

Our results demonstrated that the stimulation of MMP-1 production in HMVEC-L cells by EMMPRIN is specifically mediated by a unique epitope on EMMPRIN recognized by UM-8D6 but not HIM6.

### EXAMPLE 3

### Effects of EMMPRIN on Human Endothelial Cell Migration

The role of EMMPRIN in angiogenesis can also be directly investigated using in vitro cell migration and invasion assays. Human endothelial cells derived from primary tissue (umbilical cord) HUVEC cells were used in an in vitro system wherein endothelial cells are seeded in the top wells of the transwell system, in cell medium containing 1% FBS. In the bottom wells, culturing medium with 10% FBS will serve as a chemotactic source to induce cell migration or invasion. The top and bottom wells are separated by a membrane with pores of 8 μm in diameter. The membrane is either uncoated or coated with various extracellular matrix proteins, i.e., collagen, fibronectin, vitronectin, or Matrigel, for determining cell migration or invasion, respectively.

Materials and Methods MDA-MB-231 human breast cancer cells were purchased from ATCC (Manassas, Va.). Methods for transfection and establishment of MDA-MB-231 cells stably expressing different levels of EMMPRIN have been described previously (Tang, Y. et al. (2004) Mol. Cancer Res. 2:73-80). The cells were transfected with the cDNA corresponding to human EMMPRIN open reading frame sense (MDA MB231 S1-3) or an antisense strand of the same ORF (MDA MB231 AS1-5 and MDA MB231 AS2-5) in pcDNA3.1 TOPO vector (Invitrogen, Carlsbad, Calif.). Cells transfected wit the empty vector were used as a second control (Vector).

Endothelial cell (HUVEC) migration was evaluated using QCM™-Collagen I Quantitative cell migration assay kit (Chemicon, Temecula, Calif.). HUVEC cells (100,000 in 100 μl serum-free medium) were added to the top compartment. Serum-free media conditioned by MDA MB231 cells: WT, Vector, S1-3, AS1-5, or AS2-5 was used as the chemoattactant source in the bottom compartment of chamber. In a second experiment, anti-VEGF mAb (R&D Systems, Minneapolis, Minn.) was added into the bottom compartment at various concentrations to neutralize VEGF biological activity. Cell migration assays were carried out at 37° C. for 6 hours. Insert filters were fixed and cells remained in the top compartment were removed. Filters were stained with Gentsian violet and the number of migrated cells determined using a microscopic imaging system (Pro-Plus 3D Imaging System,).

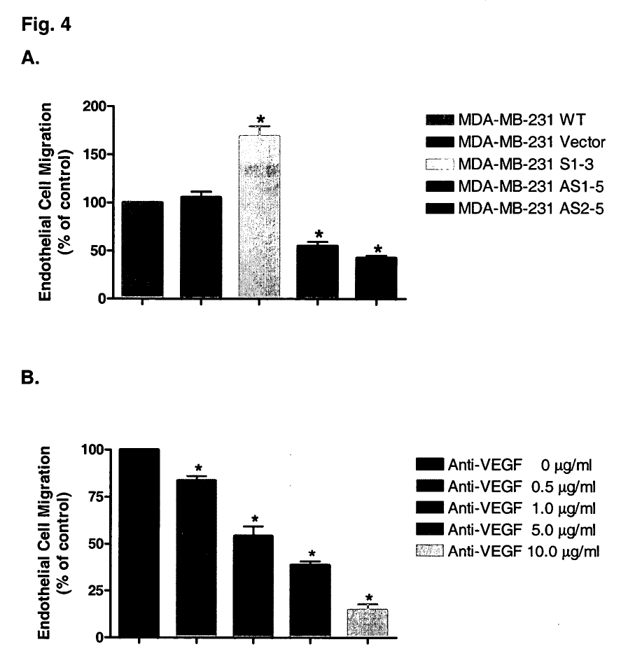

FIG. 4A shows the relative level of HUVEC cell migration induced by conditioned medium derived from the various MDA-MB-231 cell constructs. WT cell-induced migration was assigned 100%. Error bars represent standard deviations of triplicate data points. Significant differences by Students T-test (*) was at the p<0.01 value compared to endothelial cell migration induced by WT cells. FIG. 4B shows that a neutralizing antibodies to VEGF inhibited endothelial cell migration stimulated by serum-free medium conditioned by MDA-MB-231 EMMPRIN S1-3 tumor cells, assigned as 100%, in a dose-dependent manner. Error bars represent standard deviations of triplicate data points; *p<0.01 compared to endothelial cell migration in the absence of the anti-VEGF mAb.

These data demonstrate the involvement of VEGF in EMMPRIN-induced endothelial cell migration.

### EXAMPLE 4

### Effects of EMMPRIN on HMVEC-L Cell Tube Formation

The role of EMMPRIN in angiogenesis can be shown using in vitro tube formation assays. When seeded on Matrigel, HMVEC cells initiate a spontaneous differentiation process to form capillary-like tube structure. This in vitro differentiation mimics in vivo angiogenesis process and is often employed in angiogenesis studies.

We predict that EMMPRIN will change the properties of endothelial cells by stimulating MMP expression, and therefore to stimulate cell migration and invasion. An enhanced tube formation will occur when these cells are stimulated with EMMPRIN.

The specificity of EMMPRIN in tube formation will be investigated using monoclonal antibodies against human EMMPRIN.

### EXAMPLE 5

### Effects of EMMPRIN on Angiogenesis In Vivo—Matrigel Plug Assay

The role of EMMPRIN in angiogenesis will be directly investigated in vivo using Matrigel plug assays. Matrigel is a solubilized basement membrane preparation extracted from the Engel-Holm-Swarm (EHS) mouse sarcoma, a tumor rich in extracellular matrix proteins. The major component is laminin, but Matrigel also contains trace amounts of fibroblast growth factor, TGF-beta, tissue plasminogen activator, and other growth factors that occur naturally in the EHS tumor. Matrigel is the basis for several types of tumor cell invasion assays and provides the necessary substrate for the study of angiogenesis. Matrigel forms a soft gel plug when injected subcutaneously into mice or rats and supports an intense vascular response when supplemented with angiogenic factors.

Matrigel plugs containing suboptimal doses of angiogenic growth factors, such as basic fibroblast growth factor (FGF), or vascular endothelial cell growth factor (VEGF) can be implanted into mice to induce angiogenesis in vivo. Some of these plugs are supplemented with various doses of recombinant EMMPRIN. Since EMMPRIN induces endothelial cell migration and MMP production by endothelial cells, we expect to observe an increase in angiogenesis due to enhanced cell migration and invasion through Matrigel.

These effects of EMMPRIN as tested in the Matrigel plug angiogenesis assay can be used to demonstrate the activity of EMMPRIN antagonists such as siRNA or anti-EMMPRIN antibodies in preventing angiongenesis.

### EXAMPLE 6

Effects of EMMPRIN on Angiogenesis In Vivo—Corneal Pocket Assay

Similarly, the role of EMMPRIN in angiogenesis will be directly investigated in vivo using corneal pocket assays.

Polymer discs containing angiogenic growth factors, such as basic fibroblast growth factor (FGF), or vascular endothelial cell growth factor (VEGF) will be implanted into a corneal pocket in order to evoke vascular outgrowth from the peripherally located limbal vasculature. We will use a combination of suboptimal doses of angiogenic growth factors supplemented with various doses of recombinant EMMPRIN. Since EMMPRIN will induce MMP production by endothelial cells, we expect to observe an increase in angiogenesis due to enhanced endothelial cell migration and invasion.

The specificity of EMMPRIN in corneal pocket angiogenesis assay will be investigated using EMMPRIN antagonists such as siRNA or anti-EMMPRIN antibodies.

### EXAMPLE 7

### Effects of EMMPRIN on Angiogenesis—Stimulation of VEGF Production and Release Mediated by MMP

It has been reported that EMMPRIN also stimulates the expression of membrane-type matrix metalloproteinase 1 (MT1-MMP) [Sameshima et al. 2000b]. MT1-MMP in turns stimulates expression of VEGF, one of the most potent angiogenic growth factors, resulting in enhanced angiogenesis [Deryugina et al. 2002; Sounni et al. 2002]. However, the direct link between EMMPRIN and VEGF expression, and angiogenesis has yet to be established.

Using either recombinant EMMPRIN or tumor cells expressing altered levels of EMMPRIN, the link between EMMPRIN and VEGF, in both in vitro and in vivo settings can be demonstrated. As an increase in the VEGF level promotes angiogenesis, in addition to EMMPRIN-induced endothelial cell migration and invasion, the resulting effect on tumor invasiveness and growth rate is made evident.

Materials and Methods MDA-MB-231 human breast cancer cells were purchased from ATCC (Manassas, Va.). Methods for transfection and establishment of MDA-MB-231 cells stably expressing different levels of EMMPRIN have been described previously (Tang, Y. et al. (2004) Mol. Cancer Res. 2:73-80). The cells were transfected with the cDNA corresponding to human EMMPRIN open reading frame sense (MDA MB231 S1-3) or an antisense strand of the same ORF (MDA MB231 AS1-5 and MDA MB231 AS2-5) in pcDNA3.1 TOPO vector (Invitrogen, Carlsbad, Calif.).

Normal human lung or dermal fibroblast cells (NHLF or NHDF), and human microvascular endothelial cells from the lung (HMVEC-L) or human umbilical vein endothelial cells (HUVEC) were obtained from (Clonetics, Walkersville, Md.) and were cultured in Fibroblast Growth Medium or Endothelial Growth Medium-2 (EGM-2) respectively.

For co-culture studies of cancer and fibroblast cells, 100,000 cancer cells (MDI MB231 WT, S1-3, AS1-5, or AS 2-5) were cultured together with 200,000 NHDF cells in a six-well culture plate in complete DMEM. After 24 h, the medium was replaced with serum-free DMEM and the cultures continued for 2 days. The medium was replaced with fresh serum free DMEM and the cultures maintained for an additional 3 days at which time the medium was collected and analyzed. The cells were lysed with Tris-buffered saline plus 1% NP40 to determine cell-associated EMMPRIN.

The relative amount of EMMPRIN expressed in 10 ug of total cell protein was determined by Western blot analysis using scanning densitometry, by quantitative ELISA using anti-EMMPRIN antibody (RDI-147, Research diagnostics) as described (Tang et al. 2004) and on the cell surface by fluorescence activated cell analysis (FAC analysis). The FAC analysis confirmed that cell surface EMMPRIN was absent on cells transfected with antisense constructs (data not shown). The presence of MMP-2 and MMP-9 in serum-free medium or tumor extracts was determined by substrate SDS-PAGE zymography using 10 ug of total protein. Proteolytic activities on the gel were detected as clear bands on a blue background of undigested and stained gelatin. ELISA measurements of human or mouse MMP-2, MMP-9 and VEGF concentrations were performed using Quantikine ELISA kits from R&D Systems, according to the manufacturer's instructions. Each sample was analyzed in triplicates. Briefly, MMP-2, MMP-9 or VEGF contained in 100 μl of standard or samples (equivalent of 50 μg of total protein) were captured by anti-MMP-2-, anti-MMP-9-, or anti-VEGF-antibodies immobilized on the bottom of assay wells. After washing, the MMP or VEGF specific antibody was used to quantitate the amount present.

Results The transfected cells had altered levels of total EMMPRIN when grown in cell culture conditions (Table 1). S1-3 cells had approximately twice the level of WT cells and 4-fold that of the AS cells.

TABLE 1

| | EMMPRIN Expression | | VEGF |
|---|---|---|---|
| MDA MB231 cell | (Rel. Amount) | MMP Detected | (pg/ml) |
| WT | 100% | None | 208.1 |
| Vector | ND | | 175.5 |
| S1-3 | 190% | MMP-2 (weak) | 310.1 |
| AS1-5 | 47% | | 64.6 |
| AS2-5 | 62% | | 108.7 |

TABLE 2

| MDA MB231 cell | MMP Detected | VEGF (pg/ml) |
|---|---|---|
| WT | MMP-2 | 306.3 |
| | MMP-9 | |
| WT + 1.10 PA | ND | 240 |
| WT + anti-CD147 | ND | 220 |
| None (NHDF | MMP-2 | 19.5 |
| only) | | |
| S1-3 | MMP-2 | 416.1 |
| | MMP-9 | |
| AS1-5 | None | 134.7 |
| AS2-5 | None | 154.3 |

These data show a relationship between tumor cell expression of EMMPRIN, MMP expression, and VEGF levels in the engineered tumor cells alone. The co-culture data (TABLE 2) show a supra-additive amount of VEGF is produced when NHDF are present with either WT human breast tumor cells or those overexpressing EMMPRIN (S1-3).

### EXAMPLE 8

### Stimulation of In Vivo Tumor Angiogenesis by EMMPRIN

The stimulatory effects of tumor cell derived EMMPRIN on angiogenesis, the formation of new blood vessels, was directly assessed in vivo. Human breast cancer cells, MDA MB 231, were engineered to express different levels of EMMPRIN protein using recombinant DNA technology. Sense EMMPRIN cells were created that represent a cell population derived from a single cell clone that was stably transfected with a mammalian expression vector encoding the full-length human EMMPRIN. Antisense cells were generated by transfecting MDA MB 231 cells with a mammalian expression vector encoding the full-length human EMMPRIN in the antisense orientation. Sense cells constitutively express increased levels of EMMPRIN, and antisense cells express decreased levels of EMMPRIN due to inhibition of protein translation by the antisense RNA (See Example 7). These cells, together with wild-type cells, were implanted subcutaneously into nude mice. Tumor angiogenesis were assessed in tumors derived from these cells.

All procedures involving animals and their care were conducted in conformity with the company ICAUC guidelines that are in compliance with the NIH standard. Four-week-old female CD1 Nu/Nu mice were obtained from Charles River Laboratories, and acclimated for 10-14 days prior to the experiment.

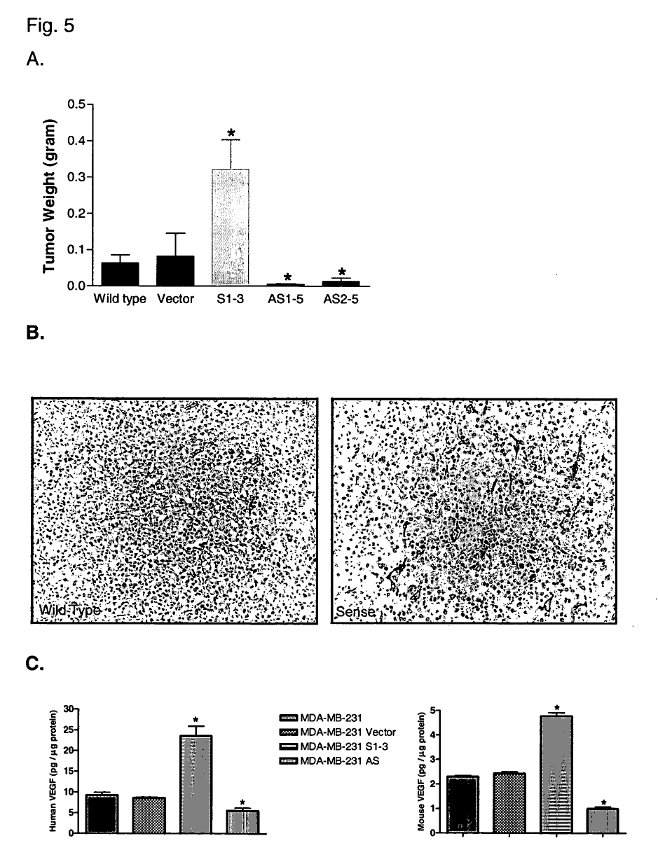

Comparing with tumors derived from wild type or vector control tumor cells, a 5-fold increase in final tumor weight was seen in S1-3 tumors produced by the EMMPRIN-overexpressing cells (FIG. 5A). AS1-5 and AS2-5 cells produced significantly smaller size (p=0.0242 and 0.0439, respectively) compared to the unaltered control cells, WT, or those transfected with empty vector, Vector, during the same period of time (FIG. 5A).

As shown in FIG. 5B, increased angiogenesis was evidenced by numerous new capillary blood vessels in tumors derived from sense cells, but not in tumors derived from wild-type and antisense cells.

Human VEGF level was 2.6-fold higher in xenografts tissue produced by S1-3 cells (235.3 pg/ug total protein) as compared to WT (92.4) and Vector control cells (86.0 pg/ug total protein (p=0.0043 sense vs wild type) (FIG. 5C). Tumor tissue from AS cells, with suppressed EMMPRIN levels had 40.4% less human VEGF or 55 pg/mg total protein (p=0.0177 compared WT).

More importantly, the impact of increased EMMPRIN level on tumor cell surface to VEGF expression in tumor tissues extended beyond tumor cells. Concomitant with stimulation of tumor VEGF production, mouse stromal VEGF production also increased in mice with S1-3 tumors. Levels of host-derived VEGF escalated 2.1-fold from 23 and 24 pg/mg total protein in wild type and vector control tumors to 48 pg/ug total protein in sense tumors (p=0.00009 sense vs WT) (FIG. 5C). Mouse tissue VEGF from mice given AS cells had less VEGF by −56.6% or 10 pg/mg total protein (p=0.00013 compared with WT).

Thus, both tumor and host cell-derived VEGF levels followed EMMPRIN-levels in EMMPRIN-modified cell derived tumors and these trends. These observations support a new paradigm in which tumor EMMPRIN mediates an active interaction between tumor and stromal compartments to stimulate VEGF production and subsequently tumor angiogenesis and growth in vivo.

### EXAMPLE 9

### Effect of Tumor EMMPRIN on Tumor Tissue Environment with Respect to MMPs and VEGF

Human breast tumor cells described in Example 3, were used to assess the effect of increased of decreased EMMPRIN on tumor tissue and tumor stroma (fibroblasts, endothelial cells, and other ancillary cells) in vivo.

On day 0, at approximately 6 weeks of age, mice were assigned to each of 5 groups consisting of 8 mice per group. Animals were inoculated with 10⁷ cells in 0.1 mL of cell suspension subcutaneously in the right flank region. Tumor growth was monitored weekly by caliper measurement and tumor volume (mm³) were calculated based on the formula [length×width×width]/2. At termination of the experiment, all animals were euthanized via CO₂ asphyxiation. Primary tumors were excised, weighed, rinsed in ice-cold PBS and processed for histological/microscopic examination. Tissue specimens and sections were also snap-frozen in liquid nitrogen for protein extraction and biochemical analysis.

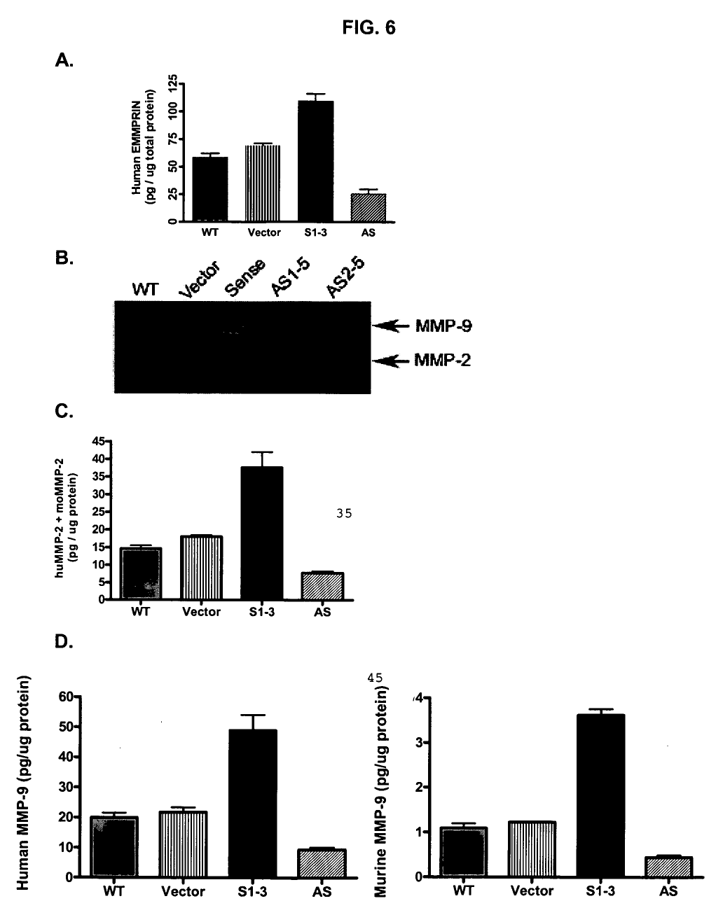

Human EMMPRIN levels were quantitatively assessed with ELISA analysis, which demonstrated a considerable gain of EMMPRIN level in sense tumors (109.8 pg/ug of total protein), and conversely a greatly suppressed level in antisense tumors (26.0 pg/ug of total protein), compared with 59.0 pg/ug of total protein in wild type tumors (p=0.000048 and 0.000077 for sense and antisense tumors vs wild type respectively) (FIG. 6A). This stable effect of EMMPRIN expression on transfected tumor cells was subsequently translated into influences on MMP expression in vivo. As expected, substrate zymography analysis of tumor tissue extracts revealed increased levels of MMP-2 and MMP-9 activities in EMMPRIN sense tumors, and lowered levels when EMMPRN expression was suppressed (FIG. 6B). MMP levels in both tumor and host compartments were then quantitatively determined by biochemical analysis. When EMMPRIN was over-expressed in tumor cells, both human MMP-2 and human MMP-9 expression levels in the resulting xenograft tumors were elevated by approximately 2.5-fold (p=0.0068 and 0.0056 compared with wild type tumors respectively) (FIG. 6C). Conversely, a 2-fold decrease in the expression of these two MMPs was observed when EMMPRIN was inhibited in antisense tumors (p=0.0026 and 0.0035 compared with wild type tumors respectively) (FIG. 6C). The effect of tumor EMMPRIN expression on host MMP-9 activity associated with stromal cells was even greater than that on tumor MMPs.

Thus, the change in tumor cell surface EMMPRIN were capable of inducing a 3.3-fold increase or a 59.3% decrease in mouse MMP-9 expression in sense or antisense tumor nodules, respectively (p=0.00013 and 0.0047 compared with wild type tumors) (FIG. 6D).

Visualization of Tumor EMMPRIN-MMP Systems In Vivo

Tumor

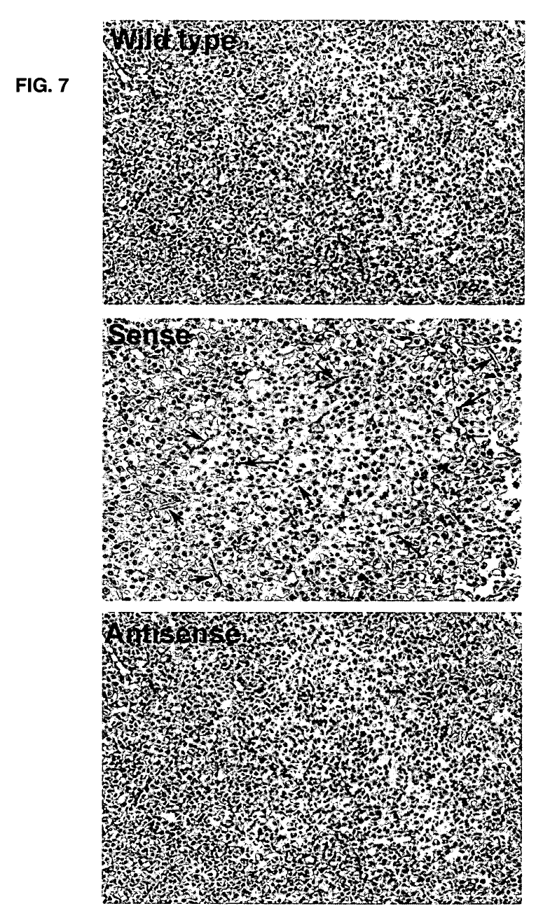

The difference in angiogenic activity is between tumors produced by the cells overexpressing EMMPRIN and the WT or under expressing cells, AS, were clearly visible (FIG. 7).

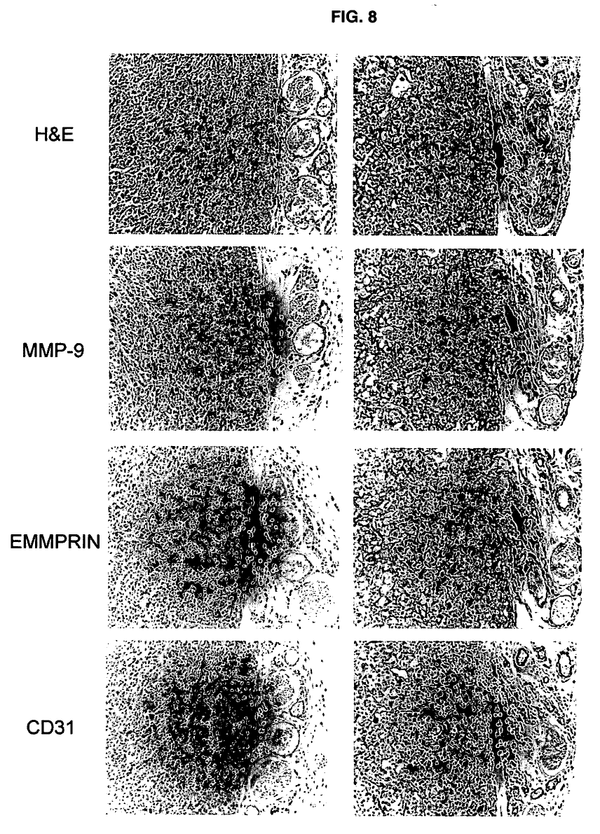

The effect of tumor EMMPRIN expression on host EMMPRIN-MMP system was further studied in immunohistochemical analysis of the xenograft tumors. In tumors produced by tumor cells that over-express EMMPRIN (MDA MB231 S1-3), up-regulation of both mouse MMP-9 and EMMPRIN was detected in stromal cells. The expression of these two proteins was restricted to mouse cells and was not detected in xenograft human tumor cells (FIG. 8). Interestingly, in addition to the staining found in capsules surrounding the tumor or in fibroblast cells in the stromal compartments infiltrated into the tumor tissues, both mouse EMMPRIN and MMP-9 were highly up-regulated around blood vessel-like structures (FIG. 8). Co-localization of MMP-9 and EMMPRIN around angiogenic blood vessels was further supported by overlapping distribution of mouse MMP-9, EMMPRIN and that of CD31, a blood vessel marker (FIG. 8). In contrast, there were only minimal levels of MMP-9 and EMMPRIN expression in tumors produced by Vector control tumor cells. In these tumors, MMP-9 was mainly detected in macrophage-like cells, and EMMPRIN was detected at very low levels in some fibroblast cells (FIG. 8).

### EXAMPLE 10

### Production and Characterization of Anti-Angiogenic Anti-EMMPRIN Monoclonal Antibody

Anti-angiogenic anti-EMMPRIN antibodies can be prepared using standard procedures and screened using the properties described herein for anti-angiogenic anti-EMMPRIN antagonists.

Materials and Methods Three 12-14 week old Balb/c mice were obtained from Charles River Laboratories. Two mice each received combination intradermal and intraperitoneal injections of 25 μg rHuEMMPRIN (R&D Systems) (12.5 μg/site) in 75 μL PBS emulsified in an equal amount of Freund's complete adjuvant on day 0, and 25 μg rHuEMMPRIN in 75 μL PBS emulsified in an equal amount of Freund's incomplete adjuvant on days 14, 28 and 51. The third mouse received an initial injection of 25 μg of rHuEMMPRIN+0.33×10⁵ U murine IFNα+0.33×10⁵ U murine IFNβ (Biosource) in 100 μl PBS administered S.Q. at the base of the tail. On days 2 and 3, the mouse received additional injections of 0.33×10⁵ U IFNα+0.33×10⁵ U IFNβ in 100 μL PBS administered S.Q. at the base of the tail. Several weeks later, the mouse was boosted with 25 μg EMMPRIN+100 μg anti-murine CD40 agonist Mab (R&D Systems) administered S.Q. at the base of the tail.

The mice were bled at various time-points throughout the immunization schedule. Blood collections were performed by retro-orbital puncture and serum was collected for titer determination by solid phase EIA. Once titer plateau was obtained, the mice received their final booster of 25 μg of EMMPRIN in PBS given intraveneously (IV). Three days later the mice were euthanized by CO₂ asphyxiation, and the spleens were aseptically removed and immersed in 10 mL cold PBS containing 100 U/mL penicillin, 100 μg/mL streptomycin, and 0.25 μg/mL amphotericin B (PBS/PSA). Lymphocytes were harvested by sterilely passing cells though a wire mesh screen immersed in cold PBS/PSA. The cells were washed once in cold PSA/PBS, counted using Trypan blue dye exclusion and resuspended in 10 mL PBS.

Characterization of Anti-Human EMMPRIN Antibodies

Enzyme immunoassays (EIAs) were used to test hybridoma cell supernatants for the presence of human anti-EMMPRIN Mabs. Briefly, plates (Nunc-Maxisorp) were coated overnight with human EMMPRIN at 1 μg/mL in PBS. After washing in 0.15 M saline containing 0.02% (w/v) Tween 20, the wells were blocked with 1% (w/v) bovine serum albumin (BSA) in PBS for 1 hr at 37° C. Undiluted hybridoma supernatants were incubated on coated plates for 1 hour at 37° C. The plates were washed and then incubated with HRP-labeled goat anti-murine IgG, Fc specific (Sigma) diluted 1:10,000 in 1% BSA/PBS for 30 minutes at 37° C. Plates were again washed then incubated for 15 minutes at RT with 100 μl/well of citrate-phosphate substrate solution (0.1 M citric acid and 0.2 M sodium phosphate, 0.01% H₂O₂, and 1 mg/mL o-phenylenediamine dihydrochloride). Substrate development was stopped by the addition of 4N sulfuric acid at 25 μL/well and the absorbance was measured at 490 nm via an automated plate spectrophotometer. All reactive hybrid cell lines were subcloned twice by limiting dilution at 1 cell/well in cloning plates. The homogeneous cell lines were cryopreserved in freezing medium (90% FBS, 10% DMSO) and stored in liquid nitrogen.

To identify the isotype of the murine anti-human EMPRIN antibodies, the Monoclonal Antibody Isotyping Kit-IsoStrip, Dipstick Format (Roche) was used as per the manufacturer instructions. Briefly, culture supernatant was diluted 1:10 in PBS and added to the development tube. The dipstick was added to the development tube and incubated at RT for approximately ten minutes. Isotypes were determined by visual assessment following incubation. A list of eighteen different hybridoma clones that secrete a murine IgG Mab that specifically binds to human EMMPRIN is shown in Table 3.

The biologic activity of recombinant EMMPIRN used as antigen protein was assayed by its ability to stimulate production of MMP-1 from EMMPRIN stimulated in fibroblast cells was performed as described (Guo, Zucker, Gordon, Toole and Biswas, (1997), J Biol Chem 272: 24-7)(24)), modified by using highly homogenous primary human fibroblast cells of less than three passages and modified stimulation conditions. Only highly pure fibroblast cells that were confirmed being negative for cytokeratin 18, cytokeratin 19, factor VII-related antigen, and alpha actin were used in the assay. The magnitude of response to EMMPRIN stimulation was dependent on the passage of fibroblast cells. Cells of earlier passages responded more potently in producing increased amounts of MMP, compared to cells that have been cultured for more than three passages. In addition, a new cell challenge method was used. Instead of adding recombinant EMMPRIN to adherent cells, we preloaded recombinant EMMPRIN in testing wells. Suspended cells were then added into these wells and were directly exposed to recombinant EMMPRIN. This new challenge procedure ensures the maximal exposure of cell surface receptors, which are likely expressed on the basolateral surfaces and could be out of access in adherent cells, to EMMPRIN for optimal assay sensitivity.

Recombinant EMMPRIN corresponding to the extracellular domain of human EMMPRIN protein was produced in NSO cells (R&D Systems, Minneapolis, Minn.). MMP-1 activity in serum-free medium conditioned by fibroblast cells treated with different amounts of recombinant EMMPRIN protein was quantitatively determined using an MMP-1 Activity Assay Kit according to product manual (R&D Systems, Minneapolis, Minn.). Briefly, MMP-1 contained in 150 μl of standards or samples was captured by anti-MMP-1 antibodies immobilized on the bottom of assay wells. Captured MMP-1 was subsequently activated 4-aminophenylmercuric acetate (APMA). MMP substrate added into each well was cleaved by activated MMP-1 and the resulting fluorescence was determined using SpectraFluor Plus Plate Reader (TECAN, Research Triangle Park, N.C.) with the following parameters: excitation wavelength at 320 nm and emission wavelength at 405 nm. To determine the inhibitory activity of anti-EMMPRIN antibodies, antibodies were added into cell culture after cells were stimulated with recombinant EMMPRIN for 15 minutes.

The panel of monoclonal antibodies were all screened for these two activities in addition to Isotyping (TABLE 3).

TABLE 3

| CNTO# | lsotype | MMP-1 | Co-culture |
|---|---|---|---|
| 1111 | IgG2bk | N | P |
| 2169 | IgG1k | N | N |
| 120 | IgG1k | N | N |
| 5125 | IgG1k | N | N |
| 627 | IgG1k | N/A | N/A |
| 828 | IgG2bk | N | N |
| 146 | IgG1k | P | P |
| 314 | IgG1k | N | P |
| 1310 | IgG1k | N | N |
| 1412 | IgG2bk | N | N/A |
| 1513 | IgG1k | N | N |
| 1611 | IgG1k | N* | N |
| 610 | IgG1k | N | N/A |
| 1134 | IgG1k | N | N/A |
| 4153 | IgG1k | N | N/A |
| 3632 | IgG1k | N | N/A |
| 1193 | IgG1k | N | N/A |
| 4161 | IgG1k | N | N/A |
| N/A (Anti- | IgG1k | p | p |
| CD147) | | | |

The antibody designated CNTO 146 met the initial selection criteria for an anti-angiongenic anti-EMMPRIN Mab.

Inhibition of MMP-2 Production in Co-Culture of Tumor Cells and Fibroblast Cells

The co-culture assay was performed as previously described above using normal human dermal fibroblasts and human melanoma tumor cells (G361) were used and either the commercial antibody RDI CD147 or CNTO 146 were added to the cultures. Three days after the last change of serum free medium, the amount of MMP-2 was quantitated. The data showed that CNTO 146 was capable of inhibiting MMP-2 production in these co-cultures as did the commercial antibody.

1. A method for treating an angiogenesis-dependent disease in a mammal in need thereof comprising administering to the mammal an EMMPRIN antagonist in an amount effective to inhibit angiogenesis in said mammal.

2. The method of claim 1 wherein the EMMPRIN antagonist is an EMMPRIN monoclonal antibody or a fragment thereof.

3. The method according to claim 2, in which the antibody fragment is an Fab, Fab′, or F(ab′)2 fragment or derivative thereof.

4. The method according to claim 2, in which the monoclonal antibody is administered intravenously.

5. The method according to claim 2, in which the monoclonal antibody is administered in the amount of from 0.05 mg/kg to 12.0 mg/kg body weight.

6. The method according to claim 2, in which the monoclonal antibody is administered in a bolus dose followed by an infusion of said antibody.

7. The method according to claim 1, in which the mammal is a human patient.

8. The method according to claim 1, in which the angiogenesis-dependent diseases is cancer.

9. The method according to claim 1, wherein the angiogenesis-dependent diseases is a disease selected from the group consisting of angioma, angiofibroma, diabetic retinopathy, premature infant's retinopathy, neovascular glaucoma, corneal disease induced by angiogenesis, involutional macula, macular degeneration, pterygium, retinal degeneration, retrolental fibroplasias, granular conjunctivitis, psoriasis, telangiectasis, pyogenic granuloma, seborrheic dermatitis, acne and arthritis.

10. The method according to claim 1, in which said angiogenesis dependent disease is an inflammatory disease selected from the group consisting of rheumatoid arthritis, macular degeneration, psoriasis, diabetic retinopathy.

11. The method according to claim 1, in which said angiogenesis dependent disease is an angiogenic skin disorder selected from the group consisting of psoriasis, venous ulcers, acne, rosacea, warts, eczema, hemangiomas, and lymphangiogenesis.

12. The method according to claim 1, in which said angiogenesis dependent disease is a disorder involving corneal or retinal neovascularization.

13. A method for inhibiting tumor growth in a mammal in need thereof comprising administering to the mammal an EMMPRIN antagonist in an amount effective to inhibit angiogenesis of the vasculature supporting the growth of said tumor.

14. A method for preventing tumor growth in a mammal in need thereof comprising administering to the mammal an EMMPRIN monoclonal antibody or fragment thereof in an amount effective to effective to inhibit angiogenesis of the vasculature supporting the growth of said tumor

15. A method for preventing metastases in a mammal in need thereof comprising administering to the mammal an EMMPRIN antagonist in an amount effective prevent metastases in said mammal.

16. A method of any of claims 1, 2, 13, 14, or 15 wherein the EMMPRIN antagonist is administered in combination with a second anti-angiogenic agent.

17. A method of claim 16 where the second anti-angiogenic agent is a Mab capable of specifically binding the adhesion molecules containing alphaV.

18. The method according to claim 2 wherein the monoclonal antibody competes for binding to human EMMPRIN with the monoclonal antibody CD147-RDI/clone UM-8D6.
